(19) United States
(12) Patent Application Publication
(10) Pub. No.: US 2022/0192704 A1
(43) Pub. Date: Jun. 23, 2022

(54) NEEDLE GUIDING ARRANGEMENT FOR IMPLANTATION OF TRANSFORMER CORE

(71) Applicant: InvivoPower AB, MOLNDAL (SE)

(72) Inventor: Bengt Bern, MÖLNDAL (SE)

(21) Appl. No.: 17/542,735

(22) Filed: Dec. 6, 2021

(30) Foreign Application Priority Data
Dec. 18, 2020 (SE) 2051504-5

(51) Int. Cl. A61B 17/34; A61M 60/873; A61B 17/04

(52) U.S. Cl. CPC A61B 17/3468; A61N 1/3787; A61B 17/0482; A61M 60/873

(57) ABSTRACT

A needle guiding arrangement, for guiding a surgical needle when applying sutures joining two skin surfaces inside an opening of a transformer core to implant the transformer core under the skin of a patient, the needle guiding arrangement comprising: a first needle guiding member; a second needle guiding member; and an alignment arrangement configured to align the first needle guiding member and the second needle guiding member on opposite sides of the opening of the transformer core along an alignment axis passing through the first needle guiding member, the opening of the transformer core and the second needle guiding member.

### FIELD OF THE INVENTION

The present invention relates to a needle guiding arrangement for implantation of a transformer core.

### BACKGROUND OF THE INVENTION

Medical devices having one or more implantable units, generally referred to as implantable medical devices, have provided a wide range of benefits to patients over recent decades. In particular, devices such as implantable hearing aids, implantable pacemakers, defibrillators, eye implants, retina implants, heart pumps, ventricular assist devices, total artificial hearts, drug delivery systems, gastric implant, nerve stimulators, brain stimulators, functional electrical stimulation devices, such as cochlear prostheses, organ assist or replacement devices, and other partially or completely-implanted medical devices, have been successful in performing life-saving and/or lifestyle enhancement functions for a number of years.

As such, the type of implantable devices and the range of functions performed thereby have increased over the years. For example, many such implantable medical devices often include one or more instruments, apparatus, sensors, processors, controllers or other functional mechanical, electrical or electronic components that are permanently or temporarily implanted in a patient to perform diagnosis, prevention, monitoring, treatment or management of a disease or injury or symptom thereof, or to investigate, replace or modify of the anatomy or of a physiological process. Many of these implantable components receive power and/or receive data and/or transmit data over a wireless transcutaneous link from and/or to external units that are part of, or operate in conjunction with, the implantable unit.

The wireless transcutaneous link is conventionally realized as an inductive link, with an external unit comprising a transmitter winding and an implantable unit comprising a receiver winding. Typically, the receiver winding is implanted below the skin, and the transmitter winding is attached to the patient skin opposite to the implanted receiver winding such that the two windings are in parallel planes on both sides (external and implantable positions) of the skin. These systems are typically referred as TET links (TET-Transcutaneous Energy Transfer). For TET links it is rather difficult to fixate and position the transmitter winding to the skin of a patient. Gluing solutions and special vests to fixate/position the transmitter winding have been tried. Especially for life sustaining applications like heart pumps, ventricular assist devices or total artificial hearts this fixation/positioning is very critical. If the transmitter winding falls off or if it is in the wrong position the transcutaneous power transfer is affected and could in worst case be life threatening for the patient.

An improved medical system, providing for an improved energy transfer between an external unit and an internal unit is described in SE 543 180and SE 543 181. In this medical system, a transformer core is arranged under the skin of the patient and an externally accessible passage is formed through the opening of the transformer core. It would be desirable to simplify the formation of this externally accessible passage.

### SUMMARY It is an object of the present invention to simplify the formation of an externally accessible passage through the opening of a transformer core arranged under the skin of a patient.

According to the present invention, it is therefore provided a needle guiding arrangement, for guiding a surgical needle when applying sutures joining two skin surfaces inside an opening of a transformer core to implant the transformer core under the skin of a patient, the needle guiding arrangement comprising: a first needle guiding member; a second needle guiding member; and an alignment arrangement configured to align the first needle guiding member and the second needle guiding member on opposite sides of the opening of the transformer core along an alignment axis passing through the first needle guiding member, the opening of the transformer core and the second needle guiding member.

The present invention is based on the realization that a suitable positioning of the sutures used for forming the passage through the transformer core is mainly dependent on the inner diameter of the transformer core, and that any guidance that the surgeon (or team of surgeons) can get for placing the sutures in relation to the transformer core may be beneficial to the result of the formation, as well as for the efficiency of the procedure.

Through embodiments of the present invention, the first and second needle guiding members may at least guide insertion of the needle at a predefined radial distance from the alignment axis, which provides for an improved and simplified formation of the passage through the transformer core.

According to embodiments, at least one of the first needle guiding member and the second needle guiding member may comprise a needle guiding structure configured to guide the surgical needle to penetrate the skin at a first penetration position and at a second penetration position spaced apart from the first penetration position; and leave the skin uncovered by the needle guiding structure along a line between the first penetration position and the second penetration position.

One or both of the first and second needle guiding members may comprise one or several needle guiding structures. Through this configuration of the needle guiding member(s), the positioning of sutures can be simplified, and removal of the needle guiding structure following formation of the suture(s) is provided for.

In embodiments, the alignment arrangement may comprise an alignment member configured to extend along the alignment axis through the opening of the transformer core when the needle guiding arrangement is in use. This configuration provides for convenient alignment of the first and second needle guiding members, and the alignment member may, in embodiments, be configured to provide additional functionalities, such as skin piercing and/or angular alignment and/or skin tensioning.

In other embodiments, the alignment arrangement may be distanced from the alignment axis, which may improve accessibility for the surgeon. In these embodiments, the needle guiding arrangement may be generally configured as a pincer, with the first and second needle guiding members at ends of arms of the pincer.

### BRIEF DESCRIPTION OF THE DRAWINGS

These and other aspects of the present invention will now be described in more detail, with reference to the appended drawings showing an example embodiment of the invention, wherein:

FIG. 1 is a schematic illustration of a transformer core arranged under the skin of a patient and an external winding passing through an externally accessible passage formed through the opening of the transformer core;

FIG. 2A is a side view schematically indicating incisions and sutures used for forming the externally accessible passage through the opening of the transformer core;

FIG. 2B is the same side view as if FIG. 2A, after formation and healing of the externally accessible passage;

FIG. 3 shows a needle guiding arrangement according to an example embodiment of the present invention with an exemplary needle guiding member configuration and an exemplary alignment arrangement configuration; FIG. 4 is an illustration of another exemplary needle guiding member configuration;

FIG. 5 is an illustration of a further exemplary needle guiding member configuration, and an exemplary alignment member; and

FIG. 6 shows a needle guiding arrangement according to an example embodiment of the present invention with an exemplary needle guiding member configuration.

### DETAILED DESCRIPTION OF EXAMPLE EMBODIMENTS

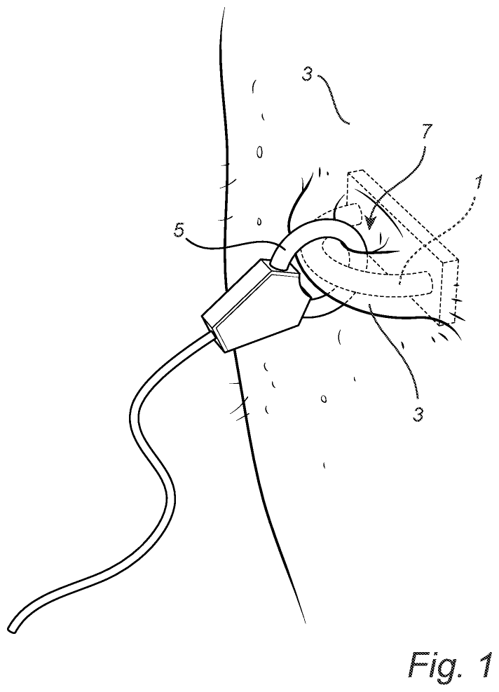

FIG. 1 is a schematic illustration of a transformer core 1 arranged under the skin 3 of a patient and an external winding 5 passing through an externally accessible passage 7 formed through the opening of the transformer core 1.

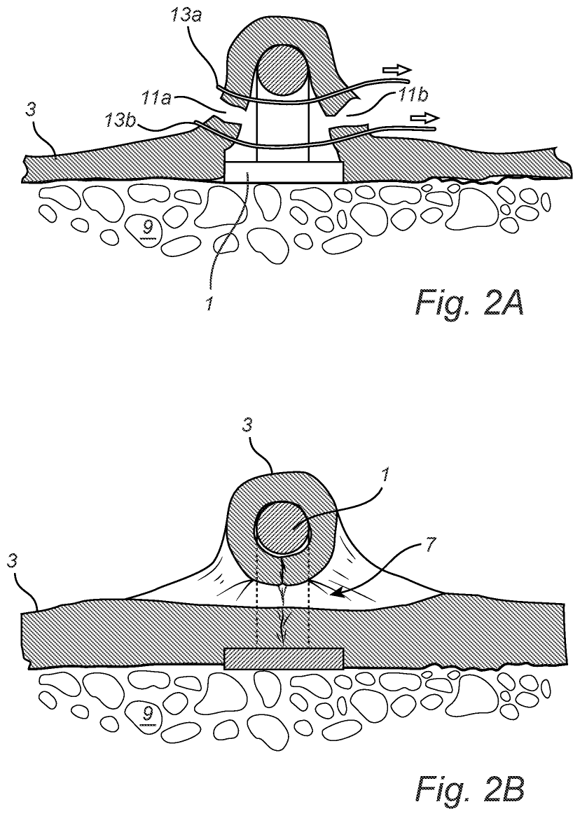

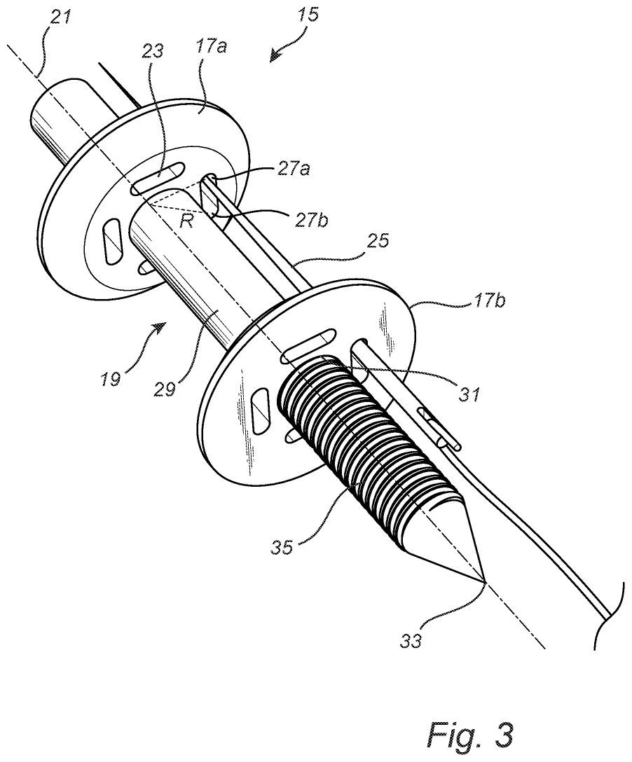

Referring now additionally to FIG. 2A, the externally accessible passage 7 is suitably formed by separating the skin 3 from the underlying tissue 9, arranging the transformer core 1 between the skin 3 and the underlying tissue 9, making first 11a and second 11b incisions through the skin 3, and connecting skin on both sides of the opening of the transformer core 1 using sutures 13a-b, preferably mattress sutures. In FIG. 2A, the sutures 13a-b are schematically indicated in an unfinished state, where they have not been tightened. Also, the relation between incisions 11a-b and the sutures 13a-b in FIG. 2A is only schematic, to aid the general understanding of the reader.

FIG. 2B is the same side view as if FIG. 2A, after formation and healing of the externally accessible passage 7 through the opening of the transformer core 1.

As should be apparent from the simple illustrations in FIGS. 2A-B, it may be cumbersome to correctly place the incisions 11a-b and the sutures 13a-b.

Various embodiments of the needle guiding arrangement according to the present invention provide for simplified and more controlled formation of the externally accessible passage 7.

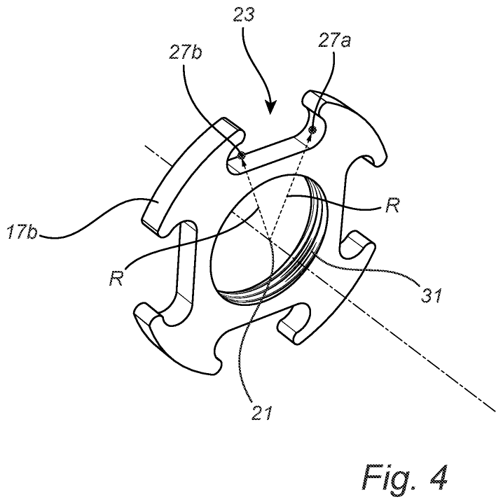

FIG. 3 shows a needle guiding arrangement 15 according to an example embodiment of the present invention. The needle guiding arrangement 15 comprises a first needle guiding member 17a, a second needle guiding member 17b, and an alignment configuration 19 configured to align the first needle guiding member 17a and the second needle guiding member 17b on opposite sides of the opening of the transformer core (not shown in FIG. 3) along an alignment axis 21 passing through the first needle guiding member 17a, the opening of the transformer core 1 and the second needle guiding member 17b.

Each of the first needle guiding member 17a and the second needle guiding member 17b of the needle guiding arrangement 15 in FIG. 3 comprises a plurality of needle guiding structures 23, here in the form of elongated openings (only one of the openings is indicated by a reference numeral in FIG. 3 to avoid cluttering the drawings). Each needle guiding structure 23 is arranged to guide a surgical needle 25 to penetrate the skin (not shown in FIG. 3) at penetration positions 27a-b that are at a predetermined radial distance R from the alignment axis 21, and to leave the skin exposed along a line between the first 27a and second 27b penetration positions. This will allow removal of the needle guiding arrangement 15 following formation of the sutures 13a-b (referring to FIG. 2A).

In the example configuration of FIG. 3, the alignment arrangement 19 comprises an alignment member 29 configured to extend along the alignment axis 21 through the opening of the transformer core (not shown in FIG. 3) when the needle guiding arrangement 15 is in use. In the example configuration of FIG. 3, the alignment member 29 is integrated with the first needle guiding member 17a, and extends through an alignment opening 31 of the second needle guiding member 17b. In FIG. 3, the alignment member 29 is provided in the form of a generally cylindrical rod-shaped member with pointed tip 33 and threads 35 along a portion of the alignment member 29. The alignment opening 31 of the second needle guiding member 17b is provided with internal threads (not visible in FIG. 3) for allowing precise variation in the distance along the alignment axis 21 between the first needle guiding member 17a and the second needle guiding member 17b.

In the example configuration of the first and second needle guiding members 17a-b in FIG. 3, both needle guiding members 17a-b are provided with needle guiding structures 23 in the form of elongated openings. However, the needle guiding structures can be provided in various other forms. For instance, referring to FIG. 4, the needle guiding structures 23 of the needle guiding member 17b has an open perimeter, while still being shaped to provide guiding support to the needle in the radial direction and in the angular direction, in respect of the alignment axis 21 at the first penetration position 27a and the second penetration position 27b. The needle guiding structure configuration in FIG. 4 does not provide quite as much needle guidance as the needle guiding structure in FIG. 3, but may provide a clearer view to the surgeon while placing the sutures.

Like the second needle guiding member in FIG. 3, the second needle guiding member 17b in FIG. 4 is provided with internal threads in its alignment opening 31. Hereby, the second needle guiding member 17b can be rotated around the alignment axis 21, while at the same time adjusting the distance between the first needle guiding member 17a and the second needle guiding member 17b. This allows for adaptation of the distance/tension between the needle guiding members 17a-b to various factors, such as the thickness of the skin 3 temporarily separated from the underlying tissue 9, as well as alignment between needle guiding structures 23 on the first 17a and second 17b needle guiding members.

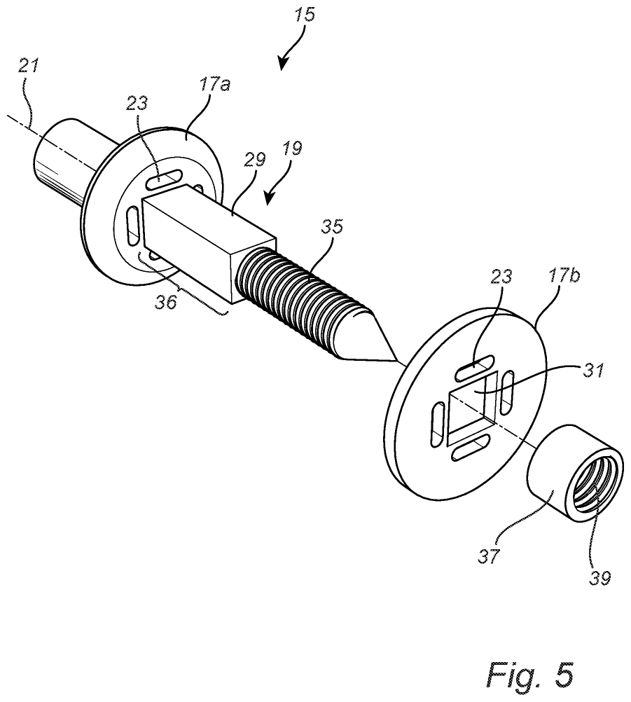

Another way of achieving such alignment will be described with reference to FIG. 5. FIG. 5 also illustrates an alternative way of allowing control of the distance along the alignment axis 21 between the first 17a and second 17b needle guiding members, while at the same time providing a clearer view for the surgeon placing the sutures.

Turning first to the issue of alignment, the alignment opening 31 of the second needle guiding member 17b in FIG. 5 is rotationally non-symmetric in respect of the alignment axis 21. Furthermore, the alignment member 29 has a portion 36 with a rotationally non-symmetric cross-section, configured to interact with the alignment opening 31 of the second needle guiding member 17b to restrict rotation of the second needle guiding member in relation to the alignment member 29 when inserted in the alignment opening 31 of the second needle guiding member 17b.

In the example configuration in FIG. 5, the distance between the first 17a and second 17b needle guiding members can obviously not be controlled by interaction between threads on the alignment member 29 and corresponding threads in the alignment opening 31 of the second needle guiding member 17b. Instead, this functionality is provided by a separate tensioning member 37 with an internal thread 39 to co-operate with the thread 35 of the alignment member 29. Without the need for threads in the alignment opening 31 of the second needle guiding member 17b, which means that the second needle guiding member 17b can be made thinner. This, in turn, provides for a clearer view of the suture for the surgeon.

In this context it should be mentioned that it is by no means necessary for both of the first needle guiding member 17a and the second needle guiding member 17b to be provided with needle guiding structures that can fully define a penetration location for the needle 25, but that one or both of the needle guiding members 17a-b could be provided as simpler structures, such as having a circular or square cross-section with a plane perpendicular to the alignment axis 21. For instance, the tensioning member 37 in FIG. 5 could individually function as a needle guiding structure defining the distance R from the alignment axis 21. A needle guiding member with a square cross-section would be able to provide additional guidance.

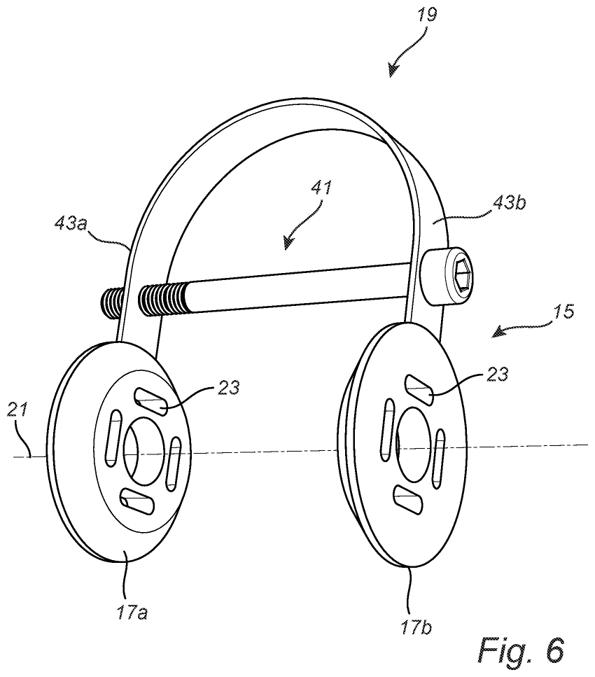

So far, various embodiments of the needle guiding arrangement 15 have been described, in which the alignment arrangement includes an alignment member 29 for passing through the opening of the transformer core 1. Another embodiment of the needle guiding arrangement 15 will now be described with reference to FIG. 6.

In the embodiment of the needle guiding arrangement 15 schematically shown in FIG. 6, the alignment arrangement 19 is distanced from the alignment axis 21, potentially allowing more unhindered access to the surgeon placing the sutures. The alignment arrangement 19 comprises a tensioning arrangement 41 for controlling the distance between the first needle guiding member 17a and the second needle guiding member 17b along the alignment axis 21. In the example configuration of FIG. 6, the alignment arrangement 19 functions as a pincer with a first arm 43a having the first needle guiding member 17a at a first end thereof and a second arm 43b having the second needle guiding member 17b at a first end thereof. The second ends of the first and second arms 43a-b are joined together, such as integrally formed or hinged. In the claims, the word “comprising” does not exclude other elements or steps, and the indefinite article “a” or “an” does not exclude a plurality. The mere fact that certain measures are recited in mutually different dependent claims does not indicate that a combination of these measured cannot be used to advantage.

What is claimed is:

1. A needle guiding arrangement, for guiding a surgical needle when applying sutures joining two skin surfaces inside an opening of a transformer core to implant the transformer core under the skin of a patient, the needle guiding arrangement comprising:
a first needle guiding member;
a second needle guiding member; and
an alignment member configured to extend through the opening of the transformer core along an alignment axis passing through the first needle guiding member, the opening of the transformer core and the second needle guiding member, when the needle guiding arrangement is in use, to thereby align the first needle guiding member and the second needle guiding member on opposite sides of the opening of the transformer core along the alignment axis.

2. The needle guiding arrangement according to claim 1, wherein at least one of the first needle guiding member and the second needle guiding member comprises a needle guiding structure configured to:
guide the surgical needle to penetrate the skin at a first penetration position and at a second penetration position spaced apart from the first penetration position; and
leave the skin exposed along a line between the first penetration position and the second penetration position.

3. The needle guiding arrangement according to claim 2, wherein the first penetration position and the second penetration position are at the same radial distance from alignment axis, and angularly spaced apart in respect of the alignment axis.

4. The needle guiding arrangement according to claim 2, wherein the needle guiding structure is shaped to provide guiding support to the needle in a radial direction and in an angular direction at the first penetration position and at the second penetration position.

5. The needle guiding arrangement according to claim 4, wherein the needle guiding structure is an opening in the needle guiding member.

6. The needle guiding arrangement according to claim 2, wherein the needle guiding member comprises a plurality of needle guiding structures, angularly spaced apart in respect of the alignment axis.

7. The needle guiding arrangement according to claim 2, wherein each of the first needle guiding member and the second needle guiding member comprises a needle guiding structure.

8. The needle guiding arrangement according to claim 1, wherein:
the alignment member is integrated with the first needle guiding member;
the second needle guiding member has an alignment opening; and
the alignment member is configured to extend along the alignment axis through the alignment opening of the second needle guiding member when the needle guiding arrangement is in use.

9. The needle guiding arrangement according to claim 8 wherein at least a portion of the alignment member is threaded to allow for adjustment of the distance along the alignment axis between the first needle guide member and the second needle guide member.

10. The needle guiding arrangement according to claim 8, wherein:
the alignment opening of the second needle guiding member is rotationally non-symmetric in respect of the alignment axis; and
the alignment member has a portion with a rotationally non-symmetric cross-section, configured to interact with the alignment opening of the second needle guiding member to restrict rotation of the second needle guiding member in relation to the alignment member when inserted in the alignment opening of the second needle guiding member.

11. The needle guiding arrangement according to claim 1, wherein an end of the alignment member is shaped for puncturing the skin.

12. The needle guiding arrangement according to claim 1, wherein the alignment arrangement comprises a tensioning arrangement for controlling a distance between the first and second needle guiding members along the alignment axis.
